(19) United States
(12) Patent Application Publication
(10) Pub. No.: US 2005/0033263 A1
(43) Pub. Date: Feb. 10, 2005

(54) System and method for restenosis mitigation

(75) Inventors: Rebecca Gottlieb, Culver City, CA (US); Bradley J. Enegren, Moorpark, CA (US); Rajiv Shah, Rancho Palos Verdes, CA (US)

Correspondence Address: FOLEY & LARDNER, 2029 CENTURY PARK EAST, SUITE 3500, LOS ANGELES, CA, 90067, US

(73) Assignee: MEDTRONIC-MINIMED, INC.

(21) Appl. No.: 10/638,215

(22) Filed: Aug. 7, 2003

(51) Int. Cl. A61M 31/00

(52) U.S. Cl. 604/508; 623/1.42

(57) ABSTRACT

A system and method for restenosis mitigation at a trauma site within the vasculature. The system may include a catheter capable of delivering a restenosis mitigating drug and a sensor extending through a lumen in the catheter. The catheter may be positioned adjacent the trauma site and the restenosis mitigating drug may be delivered to the trauma site through the catheter. The restenosis mitigating drug or some other parameter may be sensed by the sensor.

### BACKGROUND

1. Field of the Invention

The present invention relates to systems and methods for the mitigation of restenosis and, in particular embodiments, to systems and methods for the mitigation of restenosis that occurs as a result of the placement of a stent in a vein or artery.

2. Description of Related Art

Many patients who undergo procedures that induce trauma to a portion of the vasculature tend to suffer from restenosis, a narrowing or blockage of a vein or artery at the site where the trauma occurred. The development of restenosis is generally a result of alterations in endothelial healing mechanisms due to hyperglycemia, hyperinsulinemia, the ratio of glucose to insulin and the like which tend to cause an aggressive overproduction of smooth muscle cells, similar to scar tissue, at the trauma site. While the general population may suffer from restenosis following a trauma-inducing event, the incidence of restenosis is particularly high for patients whose immune system is weakened or for those who are at general disadvantage for healing, such as diabetics, for example.

The types of procedures that can induce trauma to the vasculature are varied. For example, an angioplasty procedure, in which a balloon is used to clear plaque from a blood vessel or to open a narrowing of a blood vessel, can be a trauma-inducing event. A stent procedure, in which a slotted or expandable metal tube is inserted into a blood vessel to act as a scaffold and provide structural support for the blood vessel, or a thrombolectomy, in which an instrument is used to “tunnel” through plaque or other blockage in a blood vessel, are also procedures that can induce trauma at a site in the vasculature. Regardless of the procedure inducing the trauma, however, restenosis can occur at the trauma site and cause a narrowing or blockage at that site in the blood vessel. This causes concern to medical practitioners because intervention procedures may be required to reduce or eliminate recurring blockage at the trauma site.

Various techniques have been used in an effort to mitigate restenosis. For example, for a stent procedure, one technique used is to apply a restenosis mitigating drug to the stent before insertion of the stent at the trauma site. After the stent is inserted into a vessel at a trauma site, the restenosis mitigating drug is then transferred from the stent to the vessel wall as the stent makes contact with the vessel wall. However, using this technique, the amount of restenosis mitigating drug available for delivery to the trauma site is limited to the amount of the drug that can be placed on the stent prior to insertion. In addition, there is no way to locally monitor the amount of drug actually transferred to the trauma site.

Other techniques used to mitigate restenosis include physically applying a restenosis mitigating drug to the trauma site. This technique requires a procedure to apply the drug. Using this technique, the amount of drug available for application to the site may effectively be unlimited. However, since the procedure is necessarily invasive, reapplication of the drug requires a separate procedure, which would introduce additional trauma to the same or a different site. Thus, physically applying a drug to a trauma site is limited to a “one-time” operation. For trauma sites requiring multiple deliveries or continuous delivery of drug for the mitigation of restenosis, physically applying the drug is ineffective.

### SUMMARY

It is therefore an object of embodiments of the present invention to provide systems and methods for the mitigation of restenosis. It is a further object of embodiments of the present invention to provide continuous delivery of a restenosis mitigating drug. It is yet a further object of embodiments of the present invention to locally monitor a restenosis mitigating drug at a trauma site.

A method for mitigating restenosis at a trauma site within the vasculature according to an embodiment of the present invention includes positioning a catheter adjacent the trauma site and delivering a restenosis mitigating drug to the trauma site through the catheter. A stent may be located at the trauma site. A portion of the catheter may be positioned at an interior portion of the stent.

The restenosis mitigating drug may be insulin. Moreover, the restenosis mitigating drug may be delivered upstream from the trauma site. The restenosis mitigating drug may also be dispersed to the trauma site through apertures in the catheter.

The catheter may be a balloon catheter. The balloon catheter may be coated with the restenosis mitigating drug. Furthermore, the balloon catheter may abut a wall of the vasculature at the trauma site after the balloon catheter is expanded. The restenosis mitigating drug may be transferred to the trauma site when the balloon catheter abuts the wall of the vasculature. The restenosis mitigating drug may also be dispersed to the trauma site through apertures in the balloon catheter.

A method for mitigating restenosis at a trauma site within the vasculature according to an embodiment of the present invention may also include sensing an analyte with the catheter. The delivery of the restenosis mitigating drug may be modified in response to the sensing of the analyte. The analyte may be glucose.

According to embodiments of the present invention, a flow rate of the restenosis mitigating drug may be adjusted. A dispersal pattern of the restenosis mitigating drug may also be adjusted. The restenosis mitigating drug may be nitric oxide, an antibody, a steroid, an interleukin, or a blood thinner.

The catheter may be positioned prior to or subsequent to a stent procedure.

A system for mitigating restenosis at a trauma site within the vasculature according to embodiments of the present invention may include a catheter, the catheter being capable of delivering a restenosis mitigating drug, and a sensor, the sensor extending through a lumen in the catheter. The restenosis mitigating drug may be insulin. The sensor may be a glucose sensor.

The catheter may be disposed in proximity to the trauma site. The catheter may include infusion apertures. The catheter may be a balloon catheter. The catheter may include an infusion site upstream from the trauma site. The balloon catheter may be coated with the restenosis mitigating drug.

### BRIEF DESCRIPTION OF THE DRAWINGS

FIG. 1 shows a generalized system for restenosis mitigation according to an embodiment of the present invention.

FIG. 2 shows a generalized system for restenosis mitigation according to another embodiment of the present invention.

FIG. 3A shows an infusion aperture pattern for a catheter according to an embodiment of the present invention.

FIG. 3B shows an infusion aperture pattern for a catheter according to another embodiment of the present invention.

FIG. 4 shows a generalized system for restenosis mitigation according to another embodiment of the present invention.

FIG. 5 shows a generalized method for restenosis mitigation according to another embodiment of the present invention.

### DETAILED DESCRIPTION

In the following description of preferred embodiments, reference is made to the accompanying drawings which form a part hereof, and in which are shown by way of illustration specific embodiments in which the invention may be practiced. It is to be understood that other embodiments may be utilized and structural changes may be made without departing from the scope of the preferred embodiments of the present invention.

Although the following description is directed primarily toward methods and systems for the delivery of insulin or other drugs capable of mitigating stent restenosis and for the sensing of glucose, embodiments of the present invention may be used in a variety of capacities and applications. For example, embodiments of the present invention may be used for the mitigation of restenosis resulting from any type of vasculature trauma. Also, embodiments of the present invention may be used when there is any type of manipulation in a vessel for the purpose of reinstating flow due to a restenotic episode. Generally, embodiments of the present invention may be adapted for use in any type of drug or therapy delivery system or analyte sensing system where local infusion of a drug at a trauma site is desired to promote healing.

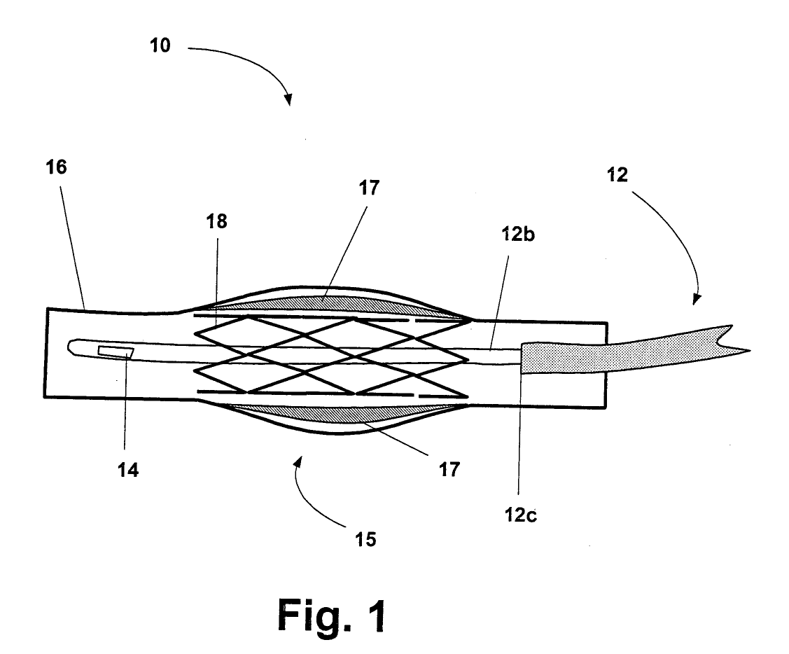

A generalized system for restenosis mitigation 10 according to an embodiment of the present invention is shown in FIG. 1. The system for restenosis mitigation 10 includes a catheter 12 capable of delivering a restenosis mitigating drug to a trauma site 15 within a vein (or artery) 16. As shown in FIG. 1, the trauma site 15 is the result of the clearing of atherosclerotic plaque 17 in the vein 16. A stent 18 has been positioned at the trauma site 15 to supply support to the vein 16. A sensing element 14 may be disposed at the end of the catheter 12.

The catheter 12 may be a multiple lumen catheter. For example, the catheter 12 may be a dual lumen catheter and thus may have a lumen for drug infusion and a lumen for a sensor. In the embodiment of the invention shown in FIG. 1, the catheter 12 is a dual lumen catheter having a sensor lumen that allows a sensor 12b to extend through the stent 18. The catheter 12 also includes a lumen for drug infusion that has an outlet site 12c that is upstream from the trauma site 15. Thus, according to the embodiment of the invention shown in FIG. 1, a restenosis mitigating drug, such as insulin, for example, can be delivered upstream from the trauma site 15 so that it flows to the trauma site 15.

A restenosis mitigating therapy may include the delivery of more than one drug to a trauma site. For example, if it is determined that two drugs should be delivered to a trauma site for effective restenosis mitigation, the catheter 12 shown in the embodiment of FIG. 1 may include three lumens, i.e., two for drug delivery and one for a sensor. In general, the catheter 12 according to the embodiment of the invention shown in FIG. 1 may include as many lumens as is desired for a therapy prescribed for the mitigation of restenosis. One embodiment of such a catheter may be seen in a patent application entitled “Multilumen Catheter,” Ser. No. 10/331,949, filed Dec. 30, 2002, and assigned to Medtronic Minimed, Inc., the contents of which are hereby incorporated by reference herein.

Various types of catheters known in the art may also be used to implement embodiments of the present invention. For example, a Swan-Ganz catheter, which has multiple catheters for injecting air, drugs and the like, may be used. Other types of catheters having one or more lumens for drug or therapy infusion, sensors and the like may also be used.

A variety of physiological, biological, biochemical, chemical or other parameters may be sensed by the sensing element 14. For example, the sensing element 14 may be a glucose sensor. If the sensing element 14 is a glucose sensor and insulin is delivered to the trauma site, the sensing element 14 may provide local sensing of the amount of insulin present at the site. By analyzing an output from the sensing element 14, the amount of insulin or other drug delivered to the site may be adjusted.

Moreover, the sensing element 14 may sense an analyte or other parameter unrelated to the drug or drugs being delivered. For example, the sensing element 14 may detect some chemical or biological property that emanates from an injured vessel or tissue. Injured tissue tends to signal for the physiological delivery of helping organisms (such as white blood cells, for example) to the trauma site where the injured tissue exists. The sensing element 14 may detect these helping organisms and an appropriate response to such detection, such as an increase in the dosage of a drug being delivered to the site, may be implemented The type of drug being delivered to a trauma site and an analyte being sensed by the sensing element 14 need not be the same.

The sensor 12 may be implanted in a variety of ways. The sensor 12 may be used for analyte sensing, physiological parameter sensing, biological parameter sensing, biochemical parameter sensing, chemical parameter sensing and the like. One embodiment of a sensor that may be used as the sensor 12 may be seen in a patent application entitled “Sensing Apparatus and Method,” Ser. No. 10/036,093, filed Dec. 28, 2001, assigned to Medtronic Minimed, Inc., the contents of which are hereby incorporated by reference herein.

The sensing element 14 may also be implanted in a variety of ways. The sensing element 14 may be a single sensing element or may be multiple sensing elements. The sensing element 14 may sense an analyte, a physiological parameter, a biological parameter, a biochemical parameter, a chemical parameter or other parameter.

If the restenosis mitigating drug delivered through the catheter is insulin, euglycemic or hypoglycemic conditions in the vicinity of the stent may be produced. In addition, if control of the insulin flow rate out of the catheter is adjusted, high local insulin concentrations at the interface between the stent and the vessel wall may be created. High local insulin levels in conjunction with nitric oxide synthase expressed from injured endothelial cells may have significant anti-proliferative effects in vitro.

The geometry of the catheter may be modified with respect to the relative locations of the sensor and infusion site to create local hypoglycemia in order to reduce platelet interaction with a freshly injured vessel wall. Decreasing the duration of platelet interaction may reduce neointimal proliferation.

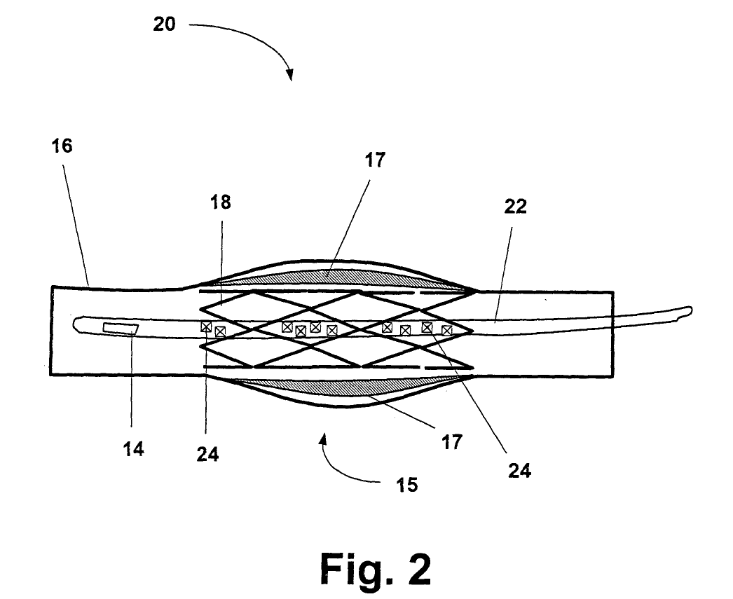

A system for restenosis mitigation 20 according to another embodiment of the present invention is shown in FIG. 2. In the embodiment of the invention shown in FIG. 2, a stent 18 has been placed at a trauma site 15 within a vein 16. Atherosclerotic plaque 17 has been cleared at the trauma site 15. A catheter 22 has been placed into the vein 16 and through the stent 18. The catheter 22 has been positioned such that a sensing element at the end of the catheter 22 resides downstream from the trauma site 15.

The catheter 22 may include infusion apertures 24 which permit infusion of a restenosis mitigating drug at the trauma site 15. Thus, according to the embodiment of the invention shown in FIG. 2, a restenosis mitigating drug can be dispersed or “sprayed” directly onto the trauma site 15 from the infusion apertures 24.

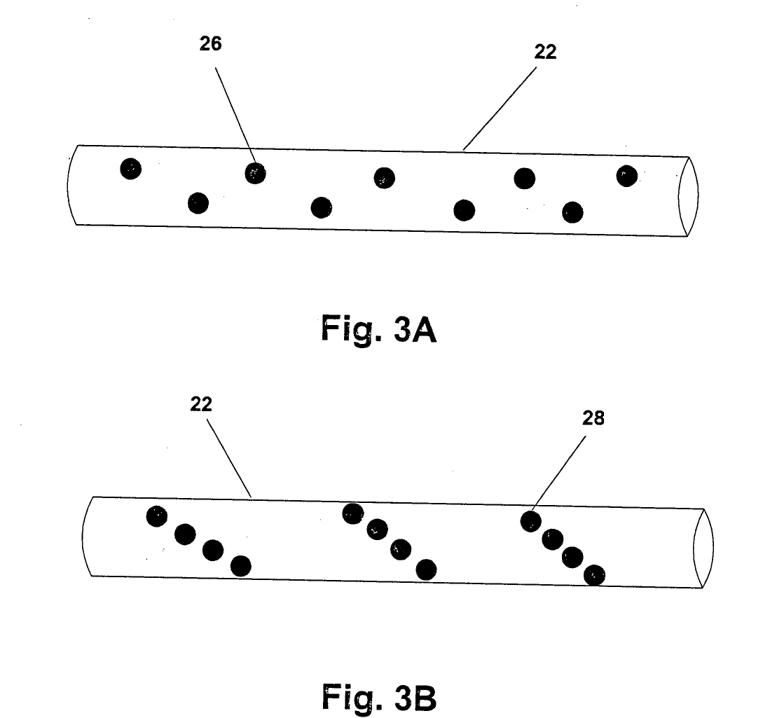

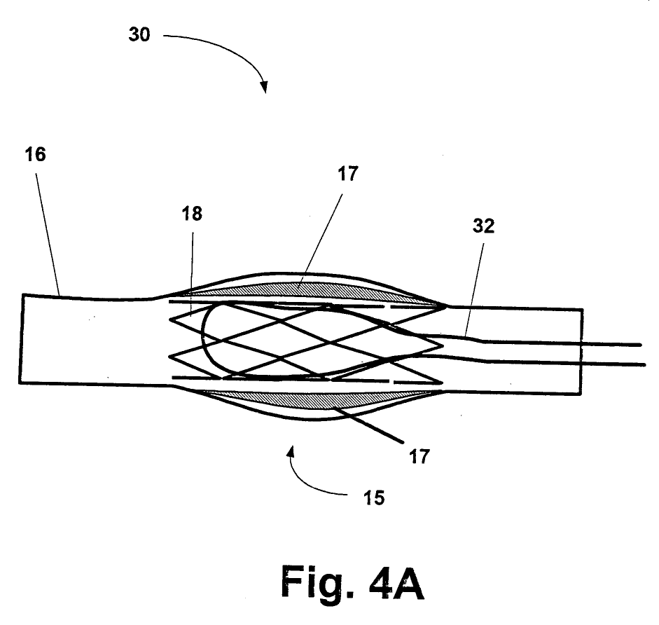

The infusion apertures 24 may be formed on the catheter 22 in a variety of ways. For example, as shown in FIG. 3A, the catheter 22 has been formed with infusion apertures 26 that have been positioned in a “zigzag” fashion. In FIG. 3B, the catheter 22 has been formed with infusion apertures 28 that spiral around the catheter 22. The particular geometry chosen for the infusion apertures 24 on the catheter 22 determines the nature of the way a restenosis mitigating drug is dispersed onto the trauma site 15. The nature of a particular restenosis may dictate that one type of dispersal pattern may be more effective than another and, thus, an infusion aperture 24 pattern for the catheter 22 may be chosen appropriately.

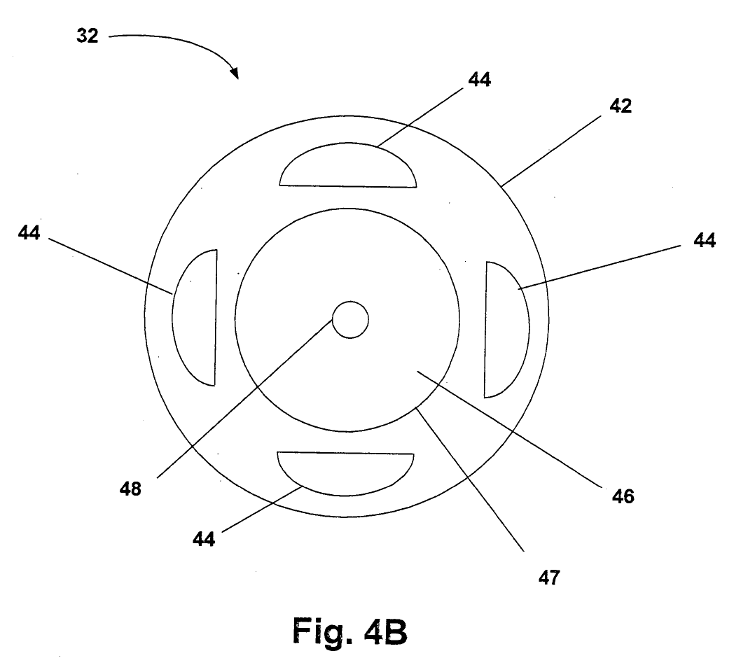

A system for restenosis mitigation 30 according to yet another embodiment of the present invention may be seen in FIG. 4A. In the embodiment of the invention shown in FIG. 4A, a stent 18 has been placed at a trauma site 15 within a vein 16. Atherosclerotic plaque 17 has been cleared at the trauma site 15. A balloon catheter 32 has been inserted into the vein 16. The end of the balloon catheter 32 is expandable and is disposed within an interior portion of the stent 18. If a restenosis mitigating drug is placed on the surface of the balloon catheter 32, the balloon catheter 32 may be expanded such that it touches the walls of the vein 16, thereby transferring the restenosis mitigating drug from the surface of the balloon catheter 32 to the trauma site 15 as the balloon catheter 32 makes contact with the wall of the vein 16 through stent 18.

The balloon catheter 32 may be formed in a variety of ways. For example, the balloon catheter 32 may be formed such that its end is expandable, as shown in the embodiment of the invention shown in FIG. 4A. In addition, the end of the balloon catheter 32 that is expandable may also be formed with infusion apertures, similar to the catheter 22 shown in FIGS. 2, 3A and 3B. Thus, a balloon catheter 32 that is expandable and includes infusion apertures may be used in a variety of ways. For example, the end of the balloon catheter 32 that is expandable may be coated with a restenosis mitigating drug. The balloon catheter 32 may then be inserted into a vessel such that the end of the balloon catheter is positioned at a trauma site. The end of the balloon catheter 22 may be expanded and deflated any number of times to transfer the restenosis mitigating drug to the vessel wall. Moreover, should additional restenosis mitigating drug be required during treatment, it may be delivered through the infusion apertures and dispersed onto the vessel wall.

If desired, the balloon catheter 32 may also include a sensing element. The sensing element may be used to sense the restenosis mitigating drug infused or titrated at a trauma site or some other physiological, biological, biochemical, chemical or other parameter.

A cutaway view of the balloon catheter 32 according to an embodiment of the present invention may be seen in FIG. 4B. An outer wall 42 of the balloon catheter 32 encompasses first drug lumens 44, an air pocket 46 having an air pocket wall 47 and a second drug lumen or sensor lumen 48. As air is forced into the air pocket 46, the air pocket wall 47 expands, pushing the first drug lumens 44 against the outer wall 42 and causing the outer wall 42 to expand. Thus, if there are infusion apertures in the first drug lumens 44 and the outer wall 42, the air forced into the air pocket 46 may cause the drug in the first drug lumens 44 to disperse onto the vessel wall. In addition, if the outer wall 42 has been coated with a restenosis mitigating drug, it may be transferred onto the vessel wall as the outer wall 42 expands in response to the expanding air pocket 46.

The types of restenosis mitigating drugs delivered by embodiments of the present invention are not limited to insulin and embodiments of the present invention are not limited to local insulin delivery. A variety of other drugs may have beneficial effects when delivered locally to a trauma site, such as, for example, nitric oxide, growth factor antibodies, steroids, interleukins, blood thinners such as coumadin or heparin and the like.

Because stents are typically placed by a balloon catheter, embodiments of the present invention may be used in conjunction with the balloon catheter used to place the stent. For example, the balloon portion of the catheter used to place the stent could be coated with a restenosis mitigating drug, such as, for example, an insulin suspended in a hydrogel. As the stent is positioned as the balloon catheter expands, the restenosis mitigating drug can be transferred to the trauma site.

Systems for restenosis mitigation according to embodiments of the present invention may be applied percutaneously. When applied percutaneously, the external portion of the catheter may be connected to a mechanism for drug infusion, control electronics and the like. Moreover, the percutaneous sites may be varied. For example, the point of entry for a system for restenosis mitigation according to embodiments of the present invention may be the subclavian vein, the internal jugular vein, ephemeral veins or any site that permits entry into the vasculature, coronary or otherwise. Systems according to embodiments of the present invention may remain in place for a few hours to a few days to several weeks, or for any length of time needed to effect the desired restenosis mitigating results.

When used in connection with a stent procedure, systems according to embodiments of the present invention may be inserted prior to or after stenting. Glucose control prior to stent placement will result in a reduction in the number and aggressiveness of circulating immune elements. Normalizing a host response system though glucose and insulin control prior to stent deployment will minimize the frequency and degree of restenosis.

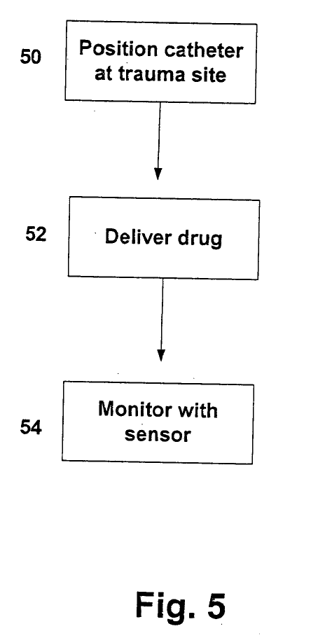

A method for restenosis mitigation may be seen in FIG. 5. At step 50, a catheter is positioned at a trauma site. At step 52, a restenosis mitigating drug is delivered through the catheter to the trauma site. The restenosis mitigating drug may be delivered upstream from the trauma site so that it flows to the site or may be dispersed to the vessel wall directly at the trauma site.

At step 54, the trauma site may be monitored with a sensor. The sensor may monitor the restenosis mitigating drug delivered through the catheter or some other physiological parameter.

While particular embodiments of the present invention have been shown and described, it will be obvious to those skilled in the art that the invention is not limited to the particular embodiments shown and described and that changes and modifications may be made without departing from the spirit and scope of the appended claims.

1. A method for mitigating restenosis at a trauma site within the vasculature comprising:
positioning a catheter adjacent the trauma site; and
delivering a restenosis mitigating drug to the trauma site through the catheter.

2. The method of claim 1, wherein a stent is located at the trauma site.

3. The method of claim 2, wherein at least a portion of the catheter is positioned at an interior portion of the stent.

4. The method of claim 1, wherein the restenosis mitigating drug is insulin.

5. The method of claim 1, wherein the restenosis mitigating drug is delivered upstream from the trauma site.

6. The method of claim 1, wherein the restenosis mitigating drug is dispersed to the trauma site through apertures in the catheter.

7. The method of claim 1, wherein the catheter is a balloon catheter.

8. The method of claim 7, further comprising disposing the restenosis mitigating drug on a balloon portion of the balloon catheter.

9. The method of claim 8, wherein the balloon catheter abuts a wall of the vasculature at the trauma site after the balloon catheter is expanded.

10. The method of claim 9, further comprising transferring the restenosis mitigating drug to the trauma site when the balloon catheter abuts the wall of the vasculature.

11. The method of claim 9, wherein the restenosis mitigating drug is dispersed to the trauma site through apertures in the balloon catheter.

12. The method of claim 1, further comprising sensing an analyte with the catheter.

13. The method of claim 12, wherein the delivery of the restenosis mitigating drug is modified in response to the sensing of the analyte.

14. The method of claim 11, wherein the analyte is glucose.

15. The method of claim 1, further comprising adjusting a flow rate of the restenosis mitigating drug.

16. The method of claim 6, further comprising adjusting a dispersal pattern of the restenosis mitigating drug.

17. The method of claim 1, wherein the catheter is positioned prior to a stent procedure.

18. The method of claim 1, wherein the catheter is positioned subsequent to a stent procedure.

19. The method of claim 1, wherein the restenosis mitigating drug is nitric oxide.

20. The method of claim 1, wherein the restenosis mitigating drug is an antibody.

21. The method of claim 1, wherein the restenosis mitigating drug is a steroid.

22. The method of claim 1, wherein the restenosis mitigating drug is an interleukin.

23. The method of claim 1, wherein the restenosis mitigating drug is a blood thinner.

24. A system for mitigating restenosis at a trauma site within the vasculature comprising:
a catheter, the catheter being capable of delivering a restenosis mitigating drug; and
a sensor, the sensor extending through a lumen in the catheter.

25. The system of claim 24, wherein the restenosis mitigating drug is insulin.

26. The system of claim 24, wherein the sensor is a glucose sensor.

27. The system of claim 24, wherein the catheter is disposed in proximity to the trauma site.

28. The system of claim 24, wherein the catheter comprises infusion apertures.

29. The system of claim 24, wherein the catheter is a balloon catheter.

30. The system of claim 24, wherein the catheter comprises an infusion site upstream from the trauma site.

31. The system of claim 29, wherein the balloon catheter is coated with the restenosis mitigating drug.

32. The system of claim 30, wherein the sensor is located downstream from the trauma site.

33. The system of claim 32, wherein a stent is located between the sensor and the infusion site.

34. The system of claim 33, wherein the sensor extends through the stent.

35. A system for mitigating stent restenosis comprising:
a stent disposed at a trauma site;
a catheter disposed adjacent the stent;
a drug for mitigating the stent restenosis; and
a sensor for monitoring the trauma site, the sensor extending through a lumen in the catheter,
wherein the catheter delivers the drug to the stent.

36. The system of claim 35, wherein the sensor senses a parameter at the trauma site.

37. The system of claim 35, wherein the sensor senses an analyte at the trauma site.

38. The system of claim 37, wherein the analyte is the drug.

39. The system of claim 36, wherein the parameter is related to the drug.

40. The system of claim 35, wherein the drug is insulin.

41. The system of claim 35, wherein sensor is a glucose sensor.

42. The system of claim 35, wherein the catheter comprises an infusion site upstream from the trauma site.

43. The system of claim 42, wherein the sensor is located downstream from the trauma site.

44. The system of claim 43, wherein a stent is disposed between the sensor and the infusion site.

45. The system of claim 44, wherein the sensor extends through the stent.

46. The system of claim 35, wherein the catheter comprises apertures.

47. The system of claim 46, wherein the apertures have a dispersal pattern.

48. The system of claim 47, wherein the drug is dispersed to the stent through the apertures.
